(19) United States
(12) Patent Application Publication
(10) Pub. No.: US 2017/0233467 A1
(43) Pub. Date: Aug. 17, 2017

(54) Antibodies Directed to Angiopoietin-1 and Angiopoietin-2 for Ocular Therapies

(71) Applicant: AMGEN INC., Thousand Oaks, CA (US)

(72) Inventors: Shu-Chen Lu, Thousand Oaks, CA (US); Murielle M. Veniant-Ellison, Thousand Oaks, CA (US); Jing Xu, Cambridge, MA (US); Hossein Salimi-Moosavi, Newbury Park, CA (US); Jonathan Daniel Oliner, Garrett Park, MD (US)

(73) Assignee: AMGEN INC., Thousand Oaks, CA (US)

(21) Appl. No.: 15/518,714

(22) PCT Filed: Oct. 16, 2015
(86) PCT No.: PCT/US2015/056091

§ 371 Date: Apr. 13, 2017

Related U.S. Application Data
(60) Provisional application No. 62/065,260, filed on Oct. 17, 2014.

(51) Int. Cl. C07K 16/22

(52) U.S. Cl. CPC C07K 16/22; C07K 2317/31; C07K 2317/76; C07K 2317/55; A61K 2039/545

(57) ABSTRACT

The present disclosure provides methods of treating ocular disorders using anti-angiogenic antibodies and pharmaceutical formulations.

The present application contains a Sequence Listing, which has been submitted electronically in ASCII format and is hereby incorporated by reference in its entirety. The computer readable format copy of the Sequence Listing, which was created on Oct. 16, 2016, is named A-1928-WO-PCT_ST25 and is 11 kilobytes in size.

### FIELD OF THE INVENTION

The field of this invention relates to methods related of treating angiogenesis related ocular disorders using antibodies that bind to angiopoietin-1 and angiopoietin-2.

### BACKGROUND OF THE INVENTION

Angiogenesis, the formation of new blood vessels from existing ones, is essential to many physiological and pathological processes. Normally, angiogenesis is tightly regulated by pro- and anti-angiogenic factors, but in the case of diseases such as cancer, ocular neovascular diseases, arthritis, and psoriasis, the process can go awry. Folkman, J., Nat. Med., 1:27-31 (1995).

Although many signal transduction systems have been implicated in the regulation of angiogenesis, one of the best-characterized and most endothelial cell-selective systems involves the Tie-2 receptor tyrosine kinase (referred to as “Tie-2” or “Tie-2R” (also referred to as “ORK”); murine Tie-2 is also referred to as “tek”) and its ligands, the angiopoietins (Gale, N. W. and Yancopoulos, G. D., Genes Dev. 13:1055-1066 [1999]). There are 4 known angiopoietins; angiopoietin-1 (“Ang-1”) through angiopoietin-4 (“Ang-4”). These angiopoietins are also referred to as “Tie-2 ligands”. (Davis, S., et al., Cell, 87:1161-1169 [1996]; Grosios, K., et al., Cytogenet Cell Genet, 84:118-120 [1999]; Holash, J., et al., Investigative Ophthalmology & Visual Science, 42:1617-1625 [1999]; Koblizek, T. I., et al., Current Biology, 8:529-532 [1998]; Lin, P., et al., Proc Natl Acad Sci USA, 95:8829-8834 [1998]; Maisonpierre, P. C., et al., Science, 277:55-60 [1997]; Papapetropoulos, A., et al., Lab Invest, 79:213-223 [1999]; Sato, T. N., et al., Nature, 375:70-74 [1998]; Shyu, K. G., et al., Circulation, 98:2081-2087 [1998]; Suri, C., et al., Cell, 87:1171-1180 [1996]; Sun, C., et al., Science, 282:468-471 [1998]; Valenzuela, D. M., et al., Proceedings of the National Academy of Sciences of the USA, 96:1904-1909 [1999]; Witzenbichler, B., et al., J Biol Chem, 273:18514-18521 [1998]). Whereas Ang-1 binding to Tie-2 stimulates receptor phosphorylation in cultured endothelial cells, Ang-2 has been observed to both agonize and antagonize Tie-2 receptor phosphorylation (Davis, S., et al., [1996], supra; Maisonpierre, P. C., et al., [1997], supra; Kim, I., J. H. Kim, et al., Oncogene 19(39): 4549-4552 (2000); Teichert-Kuliszewska, K., P. C. Maisonpierre, et al., Cardiovascular Research 49(3): 659-70 (2001)).

The phenotypes of mouse Tie-2 and Ang-1 knockouts are similar and suggest that Ang-1-stimulated Tie-2 phosphorylation mediates remodeling and stabilization of developing vessels in utero through maintenance of endothelial cell-support cell adhesion (Dumont, D. J., et al., Genes & Development, 8:1897-1909 [1994]; Sato, T. N., et al., Nature, 376:70-74 [1995]; Suri, C., et al., [1996], supra). The role of Ang-1 in vessel stabilization is thought to be conserved in the adult, where it is expressed widely and constitutively (Hanahan, D., Science, 277:48-50 [1997]; Zagzag, D., et al., Experimental Neurology, 159:391-400 [1999]). In contrast, Ang-2 expression is primarily limited to sites of vascular remodeling, where it is thought to block Ang-1 function, thereby inducing a state of vascular plasticity conducive to angiogenesis (Hanahan, D., [1997], supra; Holash, J., et al., Science, 284:1994-1998 [1999]; Maisonpierre, P. C., et al., [1997], supra).

There are a number of ocular diseases known to be associated with deregulated or undesired angiogenesis. Ocular diseases, including neovascular diabetic retinopathy, diabetic macular edema, age-related macular degeneration, are leading cause of vision loss and represent an area with high unmet medical needs. Several growth factors and their signaling pathways, including VEGF and Angiopoietins, have been implicated to play an important role in the pathogenesis of the diseases. With the success of anti-VEGF therapies in ocular diseases, drugs targeting angiopoietins may present a new promise as an improvement or adjunct to the existing therapies, and fulfill an existing unmet need.

### SUMMARY OF THE INVENTION

In one embodiment, the invention provides an improved method for treating an angiogenesis related ocular disorder in a subject, comprising administering to the subject an anti-Ang1 and/or Ang2 antibody.

In another embodiment, the invention provides a pharmaceutical formulation suitable for intraocular administration.

### BRIEF DESCRIPTION OF THE FIGURES

FIGS. 1a-c describe the results of administration of Mab-1 in an oxygen-induced retinopathy (OIR) model, demonstrating inhibition of neovascularization.

FIG. 2 describes the systemic PK profile of Mab-1 in cyno monkeys following 1 mg IV (intravenous) and 500 μg and 50 μg per eye IVT (intravitreal) doses.

FIG. 3 describes the vitreous humor and aqueous humor PK profile of Mab-1 in cyno monkeys following 500 and 50 μg doses to the vitreous humor and the aqueous humor.

FIG. 4 describes the PK profile of Mab-1 in cyno monkeys ocular tissues following 500 μg and 50 μg IVT doses.

FIG. 5 describes the predicted human ocular PK of Mab-1 following 0.5 mg/eye IVT dosing.

### DETAILED DESCRIPTION OF THE INVENTION

Angiogenesis is a biological process that involves the formation and growth of new blood vessels. Although typically a well regulated process necessary for proper development, adaptation and injury repair, there are well documented examples of angiogenesis that is associated with varying disease conditions and disorders. For example, it is clearly established that aberrant angiogenesis plays a role in tumor growth and survival. Additionally, angiogenesis is associated with certain retinopathies and other ocular disorders, rheumatoid arthritis, and psoriasis (see, e.g., Folkman, J., et al., J. Biol. Chem. 267 (1992) 10931-10934; Klagsbrun, M., et al., Annu Rev. Physiol. 53 (1991) 217-239; and Garner, A., Vascular diseases, In: Pathobiology of ocular disease, A dynamic approach, Garner, A., and Klintworth, G. K. (eds.), 2nd edition, Marcel Dekker, New York (1994), pp 1625-1710).

Accordingly, the present invention relates to methods of treating angiogenesis related ocular disorders and the use of antibodies that specifically bind to Ang1 and/or Ang2 to treat these disorders.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

Angiopoietin-1 and Angiopoietin-2 (“Ang-1,” “Ang-2,” “Ang½”) refer to the polypeptides also known as Tie-2 ligands (see, e.g., Gale, N. W. and Yancopoulos, G. D., Genes Dev. 13: 1055-1066 [1999]). Ang-2 specifically refers to the polypeptide set forth in FIG. 6 of U.S. Pat. No. 6,166,185 (“Tie-2 ligand-2”).

An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991. Alternatively, the Chothia definition, the AbM definition and the contact definition can be used to identify CDRs.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM.™., A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

An “antibody” refers to an intact immunoglobulin or to an antigen binding portion thereof that competes with the intact antibody for specific binding, unless otherwise specified. Antigen binding portions may be produced by recombinant DNA techniques or by enzymatic or chemical cleavage of intact antibodies. Antigen binding portions include, inter alia, Fab, Fab', F(ab')₂, Fv, domain antibodies (dAbs), fragments including complementarity determining regions (CDRs), single-chain antibodies (scFv), chimeric antibodies, diabodies, triabodies, tetrabodies, and polypeptides that contain at least a portion of an immunoglobulin that is sufficient to confer specific antigen binding to the polypeptide.

A Fab fragment is a monovalent fragment having the V_{L}, V_{H}, C_{L} and C_{H}1 domains; a F(ab′)₂ fragment is a bivalent fragment having two Fab fragments linked by a disulfide bridge at the hinge region; a Fd fragment has the V_{H} and C_{H}I domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. No. 6,846,634, 6,696,245, US App. Pub. No. 05/0202512, 04/0202995, 04/0038291, 04/0009507, 03/0039958, Ward et al., Nature 341:544-546 (1989)).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., Science 242:423-26 (1988) and Huston et al., 1988, Proc. Natl. Acad. Sci. USA 85:5879-83 (1988)). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48 (1993), and Poljak et al., Structure 2:1121-23 (1994)). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Complementarity determining regions (CDRs) and framework regions (FR) of a given antibody may be identified using the system described by Kabat et al. in Sequences of Proteins of Immunological Interest, 5th Ed., US Dept. of Health and Human Services, PHS, NIH, NIH Publication no. 91-3242, 1991. One or more CDRs may be incorporated into a molecule either covalently or noncovalently to make it an antibody. An antibody may incorporate the CDR(s) as part of a larger polypeptide chain, may covalently link the CDR(s) to another polypeptide chain, or may incorporate the CDR(s) noncovalently. The CDRs permit the antibody to specifically bind to a particular antigen of interest.

An antibody may have one or more binding sites. If there is more than one binding site, the binding sites may be identical to one another or may be different. For example, a naturally occurring human immunoglobulin typically has two identical binding sites, while a “bispecific” or “bifunctional” antibody has two different binding sites.

The term “human antibody” includes all antibodies that have one or more variable and constant regions characteristic of, or derived from, human immunoglobulin sequences. In one embodiment, all of the variable and constant domains are derived from human immunoglobulin sequences (a fully human antibody). These antibodies may be prepared in a variety of ways known in the art, nonlimiting examples of which are described herein, including through the immunization with an antigen of interest of a mouse that is genetically modified to express antibodies derived from human heavy and/or light chain-encoding genes.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human anti-Ang1 and/or Ang2 antibody. In another embodiment, all of the CDRs are derived from a human anti-Ang1 and/or Ang2 antibody. In another embodiment, the CDRs from more than one human anti-Ang1 and/or Ang2 antibodies are mixed and matched in a chimeric antibody. For instance, a chimeric antibody may comprise a CDR1 from the light chain of a first human anti-Ang1 and/or Ang2 antibody, a CDR2 and a CDR3 from the light chain of a second human anti-Ang1 and/or Ang2 antibody, and the CDRs from the heavy chain from a third anti-Ang1 and/or Ang2 antibody. Further, the framework regions may be derived from one of the same anti-Ang1 and/or Ang2 antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also included are fragments of such antibodies that exhibit the desired biological activity (i.e., the ability to specifically bind the human Ang1 and/or Ang2).

A “neutralizing antibody” or “inhibitory antibody” refers to an antibody that inhibits the binding of ligand to the receptor, and/or inhibits or reduces receptor signaling. The inhibition need not be complete and may be, in certain embodiments, reduced binding or signaling by at least 20%. In further embodiments, the reduction in binding or signaling is at least 30%, 40%, 50%, 60%, 70%, 80%, 85%, 90%, 95%, 97%, 99% and 99.9%. The reduced binding or signaling to Ang2 and/or Ang1 can be readily determined by one of ordinary skill in the art using conventional techniques, including, but not limited to, immunoassays.

In a specific embodiment, the antibody useful for practicing the present invention is Mab-1. See additionally, U.S. Pat. Nos. 7,521,053; 8,030,025 and 8,221,749. In a further specific embodiment, the antibody useful for practicing the present invention is any of the antibodies identified in U.S. Pat. Nos. 8,507,656; 8,133,979; 8,361,747; 8,399,626; 8,486,404; and International Patent Application Publication Nos. WO 2011/014469A1; WO 2013/112438A1; WO 2012/137993A1; WO 2013/144266A1.

### Antibody Production

The antibodies for use in the present invention can be produced by any method known in the art for the synthesis of antibodies, in particular, by chemical synthesis, hybridoma culture, or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody or a fragment of the antibody. Once a polynucleotide encoding an antibody molecule has been obtained, the vector for the production of the antibody may be produced by recombinant DNA technology. An expression vector is constructed containing antibody coding sequences and appropriate transcriptional and translational control signals. These methods include, for example, in vitro recombinant DNA techniques, synthetic techniques, and in vivo genetic recombination.

The expression vector is transferred to a host cell by conventional techniques and the transfected cells are then cultured by conventional techniques to produce an antibody of the invention. In one aspect of the invention, vectors encoding both the heavy and light chains may be co-expressed in the host cell for expression of the entire immunoglobulin molecule, as detailed below.

A variety of host-expression vector systems may be utilized to express the antibody molecules of the invention as described above. Such host-expression systems represent vehicles by which the coding sequences of interest may be produced and subsequently purified, but also represent cells which may, when transformed or transfected with the appropriate nucleotide coding sequences, express an antibody molecule of the invention in situ. Bacterial cells such as E. coli, and eukaryotic cells are commonly used for the expression of a recombinant antibody molecule, especially for the expression of whole recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, COS, 293, 3T3, or myeloma cells.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and size-exclusion chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

### Methods of Treatment

In another aspect, a method of treating a subject, comprising administering a therapeutic dosage of the antibodies of the present invention is provided. As used herein the term “subject” refers to a mammal, including humans, and is used interchangeably with the term “patient”. The anti-Ang1 and/or Ang2 antibodies, can be used to treat, control or prevent a disorder or condition characterized by undesired angiogenesis in a subject, such as ocular disorders. These ocular disorders that can be treated, prevented, or managed by methods and compositions of the present invention include but are not limited to an intraocular neovascular disease characterized by ocular neovascularization. Examples of intraocular neovascular diseases include, but are not limited to, proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, Central Retinal Vein Occlusion (CRVO), corneal neovascularization, and retinal neovascularization.

The term “treatment” encompasses alleviation or prevention of at least one symptom or other aspect of a disorder, or reduction of disease severity, and the like. An antibody according to the present invention, need not effect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method comprising administering to a patient an antibody in an amount and for a time sufficient to induce a sustained improvement over baseline of an indicator that reflects the severity of the particular disorder.

Dosages

Therapeutically effective amounts and dosages, and the frequency of administration, may vary according to such factors as the route of administration, the particular antibodies employed, the nature and severity of the disease to be treated, whether the condition is acute or chronic, and the size and general condition of the subject. Appropriate dosages can be determined by procedures known in the pertinent art, e.g. in clinical trials that may involve dose escalation studies.

The anti-Ang1 and/or Ang2 antibodies of the invention may be administered, for example, once or more than once, e.g., at regular intervals over a period of time. In particular embodiments, a human antibody is administered over a period of at least once a month or more, e.g., for one, two, or three months or even indefinitely. For treating chronic conditions, long-term treatment is generally most effective. However, for treating acute conditions, administration for shorter periods, e.g. from one to six weeks, may be sufficient. In general, the human antibody is administered until the patient manifests a medically relevant degree of improvement over baseline for the chosen indicator or indicators.

Given that the aberrant angiogenesis associated with ocular disorders occurs within the eye, achieving and maintaining a therapeutically effective dose in the eye can be challenging, and achieving a therapeutic effect given these challenges was quite unexpected. In certain embodiments, this can be achieved through intraocular administration. By administering directly to the eye, several benefits are obtained, including that the therapeutic antibody brought into close proximity of the area where angiogenesis is to be inhibited, that a higher local concentration and effective dose of antibody is achieved given the compartmentalized nature of the eye, and any potential for side effects to other parts of the body are minimized.

In certain embodiments, the intraocular administration will be given intravitreally, a specialized route of administration in which the antibody will be delivered directly into the posterior eye at the vitreous cavity. Doses for intraocular administration in these embodiments may range from 1-1000 μg per eye. In specific embodiments, the doses range from 10-1000 μg, 10-500, 10-100 or 10-50 μg per eye. In other embodiments, the doses range from 1-500, 1-100, 1-50, or 1-10 μg per eye. In further specific embodiments, the doses are 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1000 μg per eye.

Although intraocular administration is a potentially effective way of delivering the antibody, it may not be ideal from the subject's point of view. Accordingly, in other embodiments the therapeutic dose may be administered via a subcutaneous, intravenous, or intraperitoneal route. Given that the eye is somewhat of a separate compartment, achieving a therapeutically effective amount and the necessary ocular concentration of the anti-Ang1 and/or Ang2 antibody may require higher than the expected concentrations as compared to those used in, for example, an oncology indication. Accordingly, in these embodiments, the doses may range from about 0.1 mg to about 10,000 mg, e.g., about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 2000, about 3000 mg, about 4000 mg, about 5000 mg, or about 10000 mg.

Alternatively, the amount of anti-Ang1 and/or Ang2 antibody contained within the individual doses may be expressed in terms of milligrams of antibody per kilogram of subject body weight (i.e., mg/kg). For example, the antibody may be administered to a subject at a dose of about 0.0001 to about 100 mg/kg of patient body weight. In certain embodiments, the antibody may be administered at a dose of about 10 to about 50 mg/kg of body weight. In certain embodiments, the antibody may be administered at a dose of about 1, 10, 20, 30, 40, 50, 60, 70, 80, 90, 100 mg/kg of subject body weight.

Dosing frequency can range from hourly administration (e.g., a dose every hour, every 6 hours, every 12 hours, or the like) to once daily administration, weekly or monthly or even longer intervals. This dosing frequency will be determined by the skilled practitioner based on the subject's response to therapy and the desired result to be achieved, e.g., the subject's symptoms subside. Treatment may resume as needed, or, alternatively, maintenance doses may be administered as needed.

### Combination Therapies

Particular embodiments of methods and compositions of the invention involve the use of anti-ang½ antibodies in the present invention in combination with other relevant therapeutics. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which an antibody is administered at least once during a course of treatment that involves administering at least one other therapeutic agent to the patient. In certain embodiments and given the proven therapeutic use of VEGF inhibitors for angiogenesis related ocular disorders, the anti-Ang1 and/or Ang2 antibodies for use in the invention can be used in combination with VEGF inhibitors. Nonlimiting examples of additional therapeutics contemplated for combination therapies include, but are not limited to, Avastin® or Lucentis® (Genentech/Roche), or Eylea® (Regeneron), or biosimilar versions thereof.

### Efficacy Readouts and Analysis

In certain embodiments, it may be necessary to determine efficacy of the treatment with the anti-Ang1 and/or Ang2 antibody. There are several existing assays that can be used by the skilled practitioner to determine whether there is a reduction or stasis in ocular angiogenesis during and following treatment with the anti-Ang1 and/or Ang2 antibody. An exemplary assay is the counting of vascular nuclei. In certain embodiments, treatment with the anti-Ang1 and/or Ang2 antibodies for use in the invention result in a reduction of vascular nuclei of at least 10%, 20%, 30%, 40%, 50%, 60%, 70%, 80%, 90%, 100% as compared to a relevant control or untreated eye.

For a clinical setting, however, the use of in vitro assays frequently relied on would not typically be the best choice, as these often require the fixing, sectioning and staining of eye tissue, followed by examination under magnification so that vascular nuclei (e.g., endothelial and pericyte nuclei) on the vitreous side of the inner lining membrane can be counted. Alternative assays that can be used on the intact eye of a subject include using an opthalmoscope to visually examine the retina. This may optionally use the injection of a dye that enhances visualization of the ocular vasculature. Other examples include use of angiography, which is another technique that utilizes a dye injection to enhance visualization of the ocular vasculature. Additionally, slit-lamp biomicroscopy, optical coherence tomography (OCT), and microperimetry using a scanning lazer ophtalmoscope can be used.

In certain embodiments, the eyes are checked for relative pupillary afferent defect and then dilated with 2.5% phenylephrine hydrochloride and 0.8% tropicamide. Both eyes are examined using slitlamp biomicroscopy and indirect ophthalmoscopy on days before antibody injection, on days after injection, and then results are compared to evaluate treatment efficacy.

In other embodiments, fundus photography is performed on the same days as the ocular examination. Photographs may be obtained with a fundus camera (Canon Fundus CF-60Z; Canon USA Inc, Lake Success, N.Y.) and 35-mm film, but any photography device may be used and digital imaging and analysis may also be utilized.

The Imagenet Digital Angiography System (Topcon 501 A and Imagenet system; Topcon America Corp, Paramus, N.J.) may be used for fluorescein angiography. Red-free photographs of both eyes is typically obtained followed by fluorescein angiography using 0.1 mL/kg of body weight of 10% sodium fluorescein (Akorn Inc, Abita Springs, La.) at a rate of 1 mL/s. Following the fluorescein injection, a rapid series of images is obtained in the first minute of the posterior pole of first the right eye and then the left eye. Additional pairs of images are typically obtained at later intervals. Images from pre-treatment eyes can be compared to images of the eyes obtained at varying intervals post-treatment and compared to determine efficacy.

Photographs and angiograms are evaluated for evidence of excess or undesired angiogenesis, angiographic leakage, hemorrhages, or any other abnormalities. The fundus hemorrhages are graded based on a grading system with retinal hemorrhages that involves less than 3 disc areas defined as grade 1, hemorrhages between 3 and 6 disc areas defined as grade 2, and hemorrhages of more than 6 disc areas defined as grade 3. The association of hemorrhages with CNV membranes or the laser induction site is also assessed. Clinically significant bleeding is defined as any fundus hemorrhage greater than or equal to a 6-disc area.

Statistical analysis may be performed using the Population-Aggregated Panel Data with Generalized Estimating Equations and the incidence rate ratio (IRR). The incidence rate is usually defined as the number of grade 4 lesions that occur during a given interval divided by the total number of lesions induced. In phase 1, the IRR refers to the ratio of incidence rate of grade 4 lesions in the prevention eyes to the incidence rate in control eyes. An IRR of 1 signifies no difference between incidence rates. A number much smaller than 1 will indicate a reduction in the incidence of grade 4 lesions in the prevention group vs. control group. In phase 2, the incidence of grade 4 lesions in the control eyes vs. the treatment eyes is compared.

Accordingly, in certain embodiments, the invention contemplates a reduction of ocular angiogenesis as evaluated by a skilled practitioner using readily available techniques. In specific embodiments, the post-treatment eye has a reduction in incidence of graded lesions and/or grade of lesions as compared to pre-treatment. For example, a subject might have a single grade 4 lesion pre-treatment and post treatment the subject's lesion is reduced to a grade 1 lesion. Alternatively, a subject might have multiple grade 4 lesions pre-treatment and no lesions post-treatment.

### Pharmaceutical Formulations

As is understood in the pertinent field, pharmaceutical formulations comprising the antibodies of the invention are administered to a subject in a manner appropriate to the indication and the formulation. Pharmaceutical formulations may be administered by any suitable technique, including parenteral administration. If injected, the pharmaceutical formulation can be administered, for example, via intraocular, intra-articular, intravenous, intramuscular, intralesional, intraperitoneal or subcutaneous routes, by bolus injection, or continuous infusion.

The antibodies for use in the invention can be administered in the form of a formulation comprising one or more additional components such as a physiologically acceptable carrier, excipient or diluent. Optionally, the formulation additionally comprises one or more physiologically active agents, for example, as described below.

In certain embodiment, the anti-Ang1 and/or Ang2 antibodies will be administered intraocularly. Given the nature of this type of administration, there will be heightened sensitivity to discomfort by the subject receiving this injection to the eye. Accordingly, a formulation should exhibit minimal eye irritation upon and after administration during the course of treatment. Thus, in a specific embodiment, the formulation is comprised of 10 mM sodium acetate, 9% (w/v) sucrose, 0.004% polysorbate 20 and is formulated at two different pHs, 5.2 or 5.5.

In additional embodiments, the concentrations of sodium acetate can range from 1-20 mM. In further embodiments, the concentrations of sucrose can range from 1-20%. In yet further embodiments, the concentration of polysorbate 20 can range from 0.0001-0.001%. In other embodiments, the pH can range from 5.0-6.0

In accordance with appropriate industry standards, preservatives may also be added. The composition may be formulated as a lyophilizate using appropriate excipient solutions as diluents. Suitable components are nontoxic to recipients at the dosages and concentrations employed. Further examples of components that may be employed in pharmaceutical formulations are presented in Remington's Pharmaceutical Sciences, 16^{th} Ed. (1980) and 20^{th Ed. (}2000), Mack Publishing Company, Easton, Pa.

Kits for use by medical practitioners are provided including one or more antibodies of the invention and a label or other instructions for use in treating any of the conditions discussed herein. In one embodiment, the kit includes a sterile preparation of one or more of the antibodies, which may be in the form of a composition as disclosed above, and may be in one or more vials.

The invention having been described, the following examples are offered by way of illustration, and not limitation.

### EXAMPLES

### Example 1

### Inhibition of Neovascularization in OIR Models

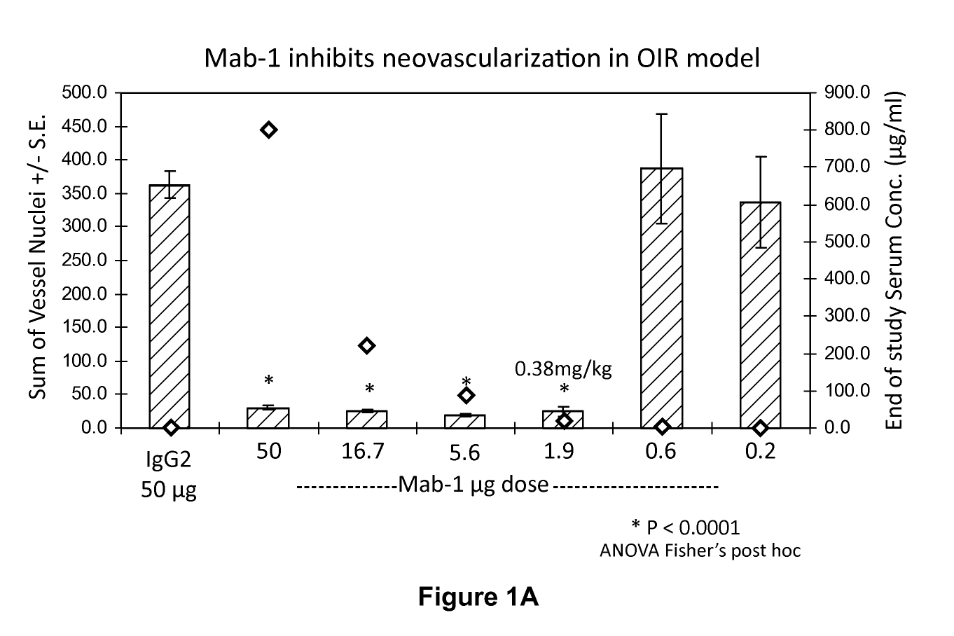

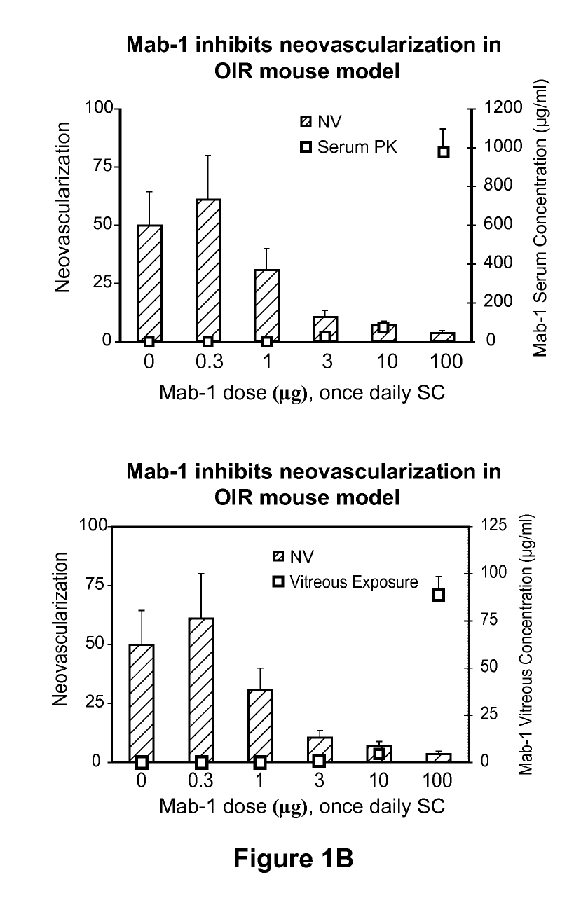

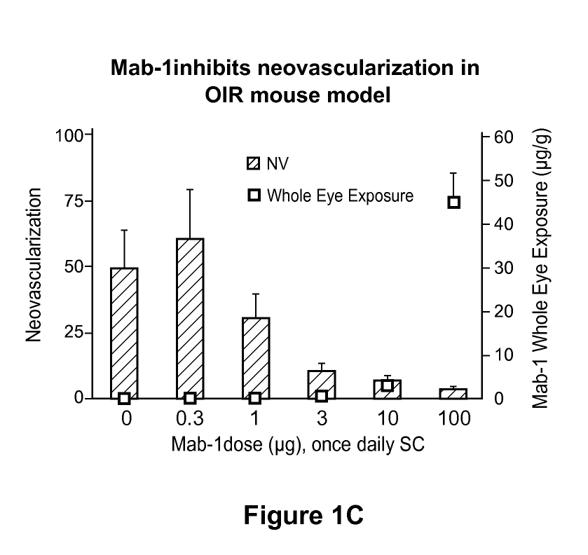

In each experiment, mice (9 mice per group) were injected with Mab-1 subcutaneously with the doses specified (FIG. 1a: 50 μg, 16.7 μg, 5.6 μg, 1.9 μg, 0.6 μg, and 0.2 μg; FIGS. 1b and 1c: 0 μg, 0.3 μg, 1 μg, 3 μg, 10 μg, 100 μg) once daily for 9 days from postnatal day 8 to day 16. A control IgG2 antibody was also administered at a 50 μg dose in the first experiment (FIG. 1a). The retina sections from each animal were used to evaluate the degree of neovascularization. The right side y-axis and blue diamonds on the graph denote the serum concentration of Mab-1 (μg/ml). The 50 μg, 16.7 μg, 5.6 μg, and 1.9 μg doses showed a marked and surprising reduction in vessel nuclei, demonstrating that an anti-Ang½ antibody can provide benefit for angiogenesis related ocular disease. This is particularly surprising, given the low end of study serum concentrations of the lower doses. FIGS. 1a-c summarize the results of these experiments.

### Example 2

### Systemic and Ocular Distribution of Mab-1 Following Intravitreal and Intravenous Administration in Cynomolgus Monkeys

### Animals: Male Cynomolgus Monkeys

Ophthalmic Exams: A board-certified veterinary ophthalmologist conducted ophthalmic exams on all available animals before dosing and on Days 4 and 15 postdose by the indirect method and by slit lamp.
Intravitreal Injections: A board-certified veterinary ophthalmologist performed the intravitreal injections.

### Study Design:

Tolerability Study (Phase 1): Two animals (both eyes) were dosed with Mab-1 formulated at pH 5.2 (A52Su) and also at pH 5.5 (A55Su)

Study Design
Group Designations and Dose Levels

| | Number | | | | | | Dose | |
|---|---|---|---|---|---|---|---|---|
| Phase/ | of Male | Dose | Target Dose | | Target Dose | | Concentration | Samples |
| Group | Animals | Route | Level | | Volume | | (mg/mL) | Collected |
| 1/1 | 2^{a} | Intravitreous^{b} | NA | | 50 | μL/eye | NA | None |
| 2/1 | 6 | Intravitreous^{c} | 500 | μg/eye | 50 | μL/eye | 10 | Blood and |
| | | | | | | | | ocular tissues |
| 2/2 | 6 | Intravitreous^{c} | 50 | μg/eye | 50 | μL/eye | 1 | Blood and |
| | | | | | | | | ocular tissues |
| 2/3 | 3 | Intravenous | 1000 | μg/animal | 1 | mL/animal | 1 | Blood |

NA Not applicable.
Note:
Extra animals may be dosed for use as replacements if applicable.
^{a}One animal dosed in Phase 1 will be the same animal used for Phase 2 Group 3. The other animal will be an additional (naïve) animal and may be transferred to the Covance stock colony after use.
^{b}Animals will receive a single administration of Vehicle 1 in the right eye and Vehicle 2 in the left eye.
^{c}Each animal will receive a single administration of Mab-1 in each eye.

Groups 1 thru 2: Blood was collected from animals at predose and at 0.083, 0.5, 2, 4, 8, 24, 96, 168, 334, and 504 hours postdose . Both eyes were collected from one animal per time point per group at 4, 24, 96, 168, 334, and 504 hours postdose. Following sacrifice, the globe was excised and the following tissues were collected. Each sample was weighed.

| | |
|---|---|
| Aqueous Humor | Retina |
| Vitreous Humor | Sclera |

Group 3: Blood (approximately 1.5 mL) was collected from each animal at predose and at 0.083, 0.5, 1, 2, 4, 8, 24, 96, 168, 334, and 504 hours postdose. Following the last blood collection the animals were sacrificed, the globe was excised and the following tissues were collected. Each sample was weighed.

| | |
|---|---|
| Aqueous Humor | Retina |
| Vitreous Humor | Sclera |

Sample Analysis: Blood was processed to obtain serum.

| Animal | | Study Day | | |
|---|---|---|---|---|
| Number | | 2 | 4 | 8 |
| I04850 | Right eye | Trace aqueous flare, mild (1+) aqueous | Trace aqueous cell, | No observation |
| | | cell which was predominantly white, mild | predominantly white | |
| | | (1+) conjunctival hyperemia | | |
| | Left eye | Trace aqueous flare, mild (1+) aqueous | Mild (1+) aqueous cell, | No observation |
| | | cell which was predominantly white, mild | predominantly white | |
| | | (1+) conjunctival hyperemia | | |
| I04851 | Right eye | Trace aqueous flare, trace aqueous cell | No observations | No observation |
| | | which was predominantly white, mild | | |
| | | (1+) conjunctival hyperemia | | |
| | Left eye | Mild (1+) aqueous flare, trace aqueous | Trace aqueous cell, | No observation |
| | | cell which was predominantly white, mild | predominantly white | |
| | | (1+) conjunctival hyperemia | | |

Right received vehicle 1 pH 5.2
Left eye received vehicle 2 pH 5.5

The Mab-1 formulation in A52Su was well tolerated in cyno intravitreal injection. A52Su was marginally better tolerated than A55Su.

### Example 3

PK Profile of Mab-1 in Cyno Serum Following 1 mg IV (intravenous) and IVT (intravitreal) Dosing

In this next experiment to assess PK profile, cynos were dosed in a manner as described in Example 2 according to the dosing plan described in Example 2. The following table shows the systemic PK attributes of Mab-1 in three different dosing groups:

Compound

| | IVT Dose | T_{1/2} | Cmax | CL/F | AUC_{0-inf} |
|---|---|---|---|---|---|
| [symbol] mpound | (mg/kg) | (h) | (μg/mL) | (mL/h/kg) | (μg · h/mL) |
| [symbol] Cyno-IV (1 mg) | 0.333 | 42 | 10.6 | 0.826 | 411 |
| [symbol] -Cyno#IVT500 | 0.333 | 112 | 1.6 | 0.950 | 350 |
| [symbol] -Cyno#IVT50 | 0.033 | 231 | 0.1 | 1.031 | 32 |

[symbol]

indicates data missing or illegible when filed

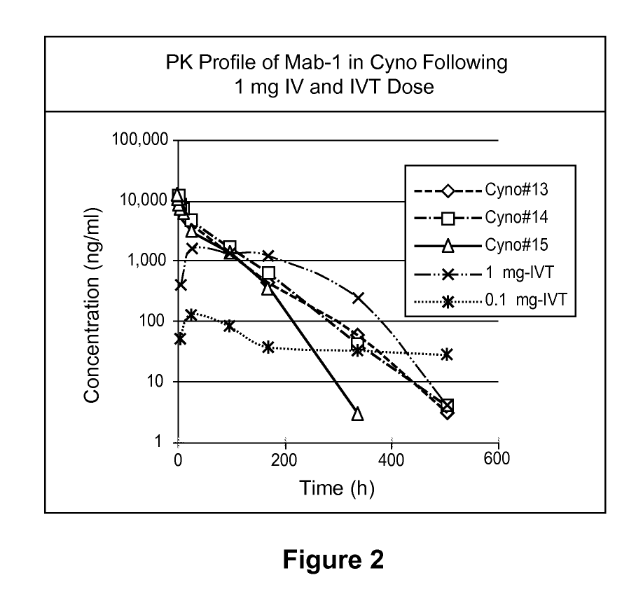

FIG. 2 summarizes the results. Mab-1 systemic clearance in 500 μg IVT group accelerated after two weeks due to ADA response. Mab-1 has longer systemic terminal T½ in 50 μg IVT group as compared to the 500 μg IVT group. No ADA in 50 μg IVT group.

### Example 4

PK Profile of Mab-1 In Cyno Vitreous and Aqueous Humor Following 500 & 50 μg/Eye IVT Dosing

Cynos were dosed as in a manner described in Example 2. The following table shows the vitreous and aqueous humor PK attributes of Mab-1 in three different dosing groups:

| | IVT Dose | T_{1/2} | Cmax | CL/F | AUC_{0-inf} |
|---|---|---|---|---|---|
| Compound | (μg/eye) | (h) | (μg/mL) | (mL/h) | (μg · h/mL) |
| Cyno#IVT50- | 50 | 144 | 22 | 0.019 | 2,624 |
| Vitreous | | | | | |
| Cyno#IVT500- | 500 | 91 | 317 | 0.019 | 25,904 |
| Vitreous | | | | | |
| Cyno#IVT50-AQ | 50 | 127 | 14 | 0.070 | 714 |
| Humor | | | | | |
| Cyno#IVT500-AQ | 500 | 75 | 70 | 0.077 | 6,484 |
| Humor | | | | | |

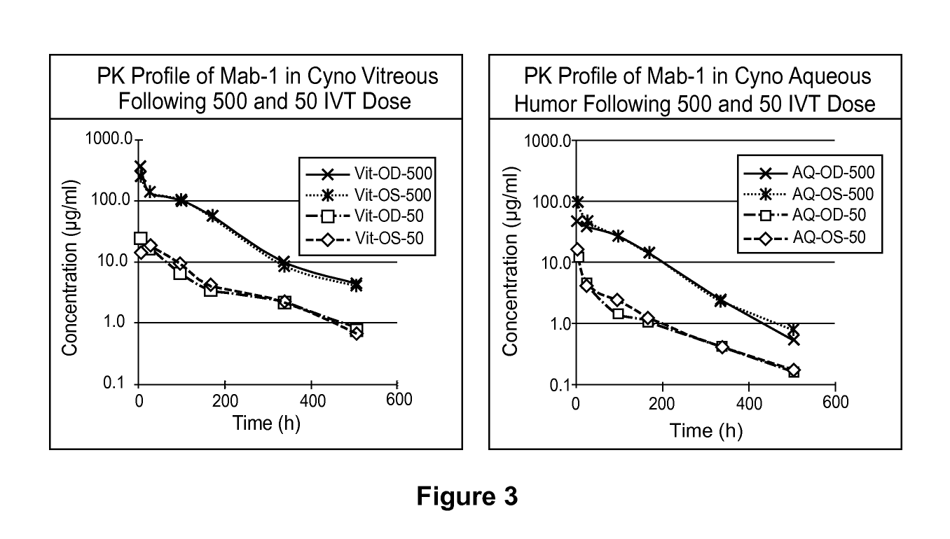

FIG. 3 summarizes the results. Longer vitreous and aqueous humor (AQ) terminal T½ in 50 μg IVT group as compared to 500 μg IVT group. Also, Mab-1 was rapidly distributed to Aqueous Humor following IVT Injection (Tmax<4 hr)

### Example 5

### Ocular Tissue Exposure of Mab-1 In Cyno Following 500 & 50 μg/Eye IVT Dosing

Cynos were dosed as in a manner described in Example 2. The following table shows the ocular tissues (retina, choroid, and sclera) PK attributes of Mab-1 in three different dosing groups:

Summary Table/Average

| | IVT Dose | T_{1/2} | Cmax | MRT | CL/F | Vz/F | Vss | AUC₀₋ₜ | AUC_{0-inf} |
|---|---|---|---|---|---|---|---|---|---|
| Compound | (μg/eye) | (h) | (μg/g) | (h) | (g/h) | (g) | (g) | (μg · h/g) | (μg · h/g) |
| Cyno#IVT50-Retina | 50 | 128 | 17 | 165 | 0.027 | 5.1 | | 1,831 | 1,932 |
| Cyno#IVT500-Retina | 500 | 101 | 129 | 131 | 0.030 | 4.3 | | 16,404 | 16,914 |
| Cyno#IVT50-Choroid | 50 | 105 | 8 | 134 | 0.063 | 9.6 | | 772 | 796 |
| Cyno#IVT500-Choroid | 500 | 97 | 34 | 148 | 0.093 | 13.1 | | 5,222 | 5,382 |
| Cyno#IVT50-Sclera | 50 | 126 | 3 | 169 | 0.157 | 29.0 | | 310 | 329 |
| Cyno#IVT500-Sclera | 500 | 82 | 19 | 134 | 0.155 | 18.3 | | 3,174 | 3,228 |

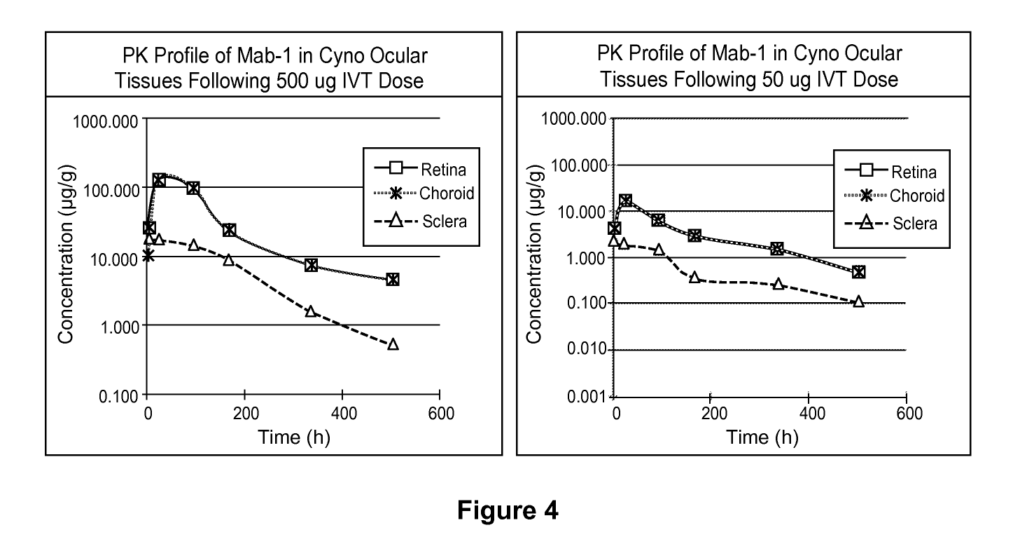

FIG. 4 summarizes the results. Exposures of Mab-1 in the retina appeared to be similar to that in the choroid, and exposures of Mab-1 in both the retina and choroid were greater than that in the sclera. Mab-1 was rapidly distributed to Retina and Choroid following IVT injection (Tmax˜24 hr).

### Example 6

### Predicted Ocular PK Profile of Mab-1 in human

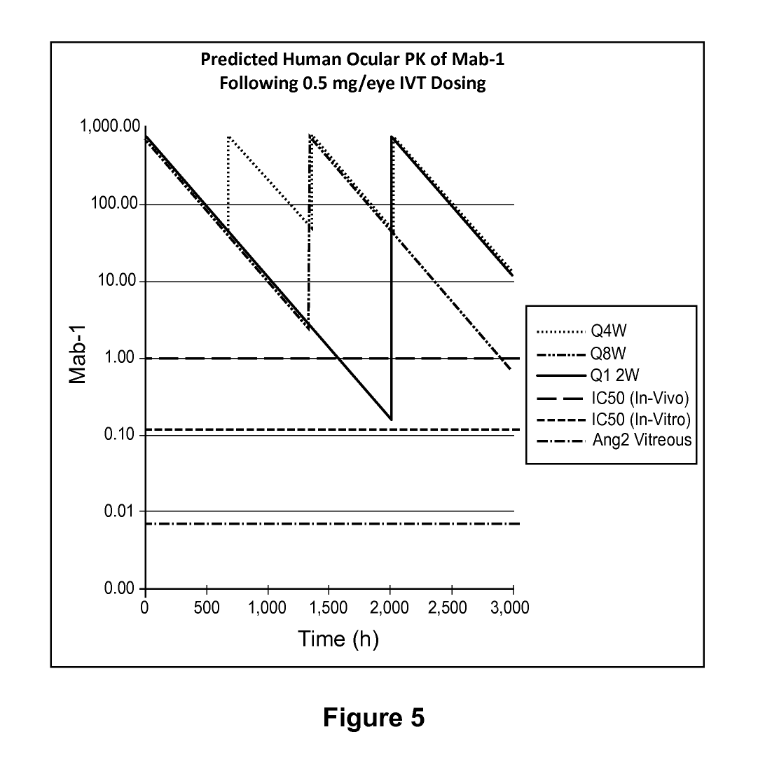

The prediction of ocular PK profile of Mab-1 in humans was performed based on the cyno vitreous clearance (0.019 mL/h) and human physiological vitreous volume (4.5 mL). FIG. 5 summarizes the predicted human ocular PK following 0.5 mg/eye IVT dosing.

Mab-1 Antibody Sequences

Mab-1 Light Chain CDR Amino Acid Sequences:
CDR-1:
(SEQ ID NO: 1)
RSSQSLLHSHGYNYLD
CDR-2:
(SEQ ID NO: 2)
LGSNRAS
CDR-3:
(SEQ ID NO: 3)
MQGTHWPPT
Mab-1 Heavy Chain CDR Amino Acid Sequences:
CDR-1:
(SEQ ID NO: 4)
SYGMH
CDR-2:
(SEQ ID NO: 5)
YISSSGSTIYYADSVKG
CDR-3:
(SEQ ID NO: 6)
DLLDYDLLTGYGY
Mab-1 Variable Light Chain Amino Acid Sequence
(SEQ ID NO: 7):
DIVMTQSPLSLPVTPGEPASISCRSSQSLLHSHGYNYLDWYLQKPGQSPQ
LLIYLGSNRASGVPDRFSGSGSGTDFTLKISRVEAEDVGVYYCMQGTHWP
PTFGQGTKLEIK
Mab-1 Variable Heavy Chain Amino Acid Sequence
(SEQ ID NO: 8):
EVQLVQSGGGVVQPGRSLRLSCAASGFTFSSYGMHWVRQAPGKGLEWVSY
ISSSGSTIYYADSVKGRFTISRDNAKNSLYLQMNSLRAEDTAVYYCARDL
LDYDLLTGYGYWGQGTLVTVSS
Mab-1 Light Chain Amino Acid Sequence
(SEQ ID NO: 9):
MDMRVPAQLLGLLLLWLRGARCDIVMTQSPLSLPVTPGEPASISCRSSQS
LLHSHGYNYLDWYLQKPGQSPQLLIYLGSNRASGVPDRFSGSGSGTDFTL
KISRVEAEDVGVYYCMQGTHWPPTFGQGTKLEIKRTVAAPSVFIFPPSDE
QLKSGTASVVCLLNNFYPREAKVQWKVDNALQSGNSQESVTEQDSKDSTY
SLSSTLTLSKADYEKHKVYACEVTHQGLSSPVTKSFNRGEC
Mab-1 Heavy Chain Amino Acid Sequence
(SEQ ID NO: 10):
MDMRVPAQLLGLLLLWLRGARCEVQLVQSGGGVVQPGRSLRLSCAASGFT
FSSYGMHWVRQAPGKGLEWVSYISSSGSTIYYADSVKGRFTISRDNAKNS
LYLQMNSLRAEDTAVYYCARDLLDYDLLTGYGYWGQGTLVTVSSASTKGP
SVFPLAPCSRSTSESTAALGCLVKDYFPEPVTVSWNSGALTSGVHTFPAV
LQSSGLYSLSSVVTVPSSNFGTQTYTCNVDHKPSNTKVDKTVERKCCVEC
PPCPAPPVAGPSVFLFPPKPKDTLMISRTPEVTCVVVDVSHEDPEVQFNW
YVDGVEVHNAKTKPREEQFNSTFRVVSVLTVVHQDWLNGKEYKCKVSNKG
LPAPIEKTISKTKGQPREPQVYTLPPSREEMTKNQVSLTCLVKGFYPSDI
AVEWESNGQPENNYKTTPPMLDSDGSFFLYSKLTVDKSRWQQGNVFSCSV
MHEALHNHYTQKSLSLSPGK

Each reference cited herein is hereby incorporated by reference in its entirety for all that it teaches and for all purposes.

The present invention is not to be limited in scope by the specific embodiments described herein, which are intended as single illustrations of individual aspects of the invention, and functionally equivalent methods and components are invention. Indeed, various modifications of the invention, in addition to those shown and described herein will become apparent to those skilled in the art from the foregoing description and accompanying drawings. Such modifications are intended to fall within the scope of the appended claims.

What is claimed is:

1. A method for treating an angiogenesis related ocular disorder in a subject, comprising administering to the subject an antibody that binds and neutralizes Ang1 and/or Ang2.

2. The method of claim 1, wherein the antibody binds and neutralizes Ang1 and Ang2.

3. The method of claim 2, wherein the administration is intraocular.

4. The method of claim 3, wherein the administration is intravitreal.

5. The method of claim 1, wherein the administration is intravenous.

6. The method of claim 1, wherein the antibody is a full length intact immunoglobulin.

7. The method of claim 1, wherein the antibody is an antibody fragment.

8. The method of claim 1, wherein the antibody is a Fab antibody fragment.

9. The method of claim 1, wherein the antibody is Mab-1.

10. The method of claim 3, wherein the antibody is administered at a dose of between 50 and 500 μg per eye.

11. The method of claim 10, wherein the antibody is administered at a dose of 50 μg per eye.

12. The method of claim 10, wherein the antibody is administered once daily for at least 7 days.

13. The method of claim 12, wherein the antibody is administered once daily for 9 days.

14. A pharmaceutical formulation suitable for intraocular administration, wherein said formulation comprises an antibody that binds and neutralizes Ang1 and/or Ang2, sodium acetate, sucrose and polysorbate-20.

15. The formulation of claim 14, wherein said pH is 5.0 to 6.0.

16. The formulation of claim 15, wherein said pH is 5.2 or 5.5.

17. The formulation of claim 16, wherein said sodium acetate concentration is 10 mM, said sucrose concentration is 9%, and said polysorbate-20 concentration is 0.0004%.

18. The formulation of claim 14, wherein said antibody binds and neutralizes Ang1 and Ang2.


SEQUENCE LISTING (10 sequences)

SEQ ID NO: 1 - protein, 16 aa, Artificial Sequence
  Mab-1 Light Chain CDR Amino Acid Sequence
RSSQSLLHSHGYNYLD

SEQ ID NO: 2 - protein, 7 aa, Artificial Sequence
  Mab-1 Light Chain CDR Amino Acid Sequence
LGSNRAS

SEQ ID NO: 3 - protein, 9 aa, Artificial Sequence
  Mab-1 Light Chain CDR Amino Acid Sequence
MQGTHWPPT

SEQ ID NO: 4 - protein, 5 aa, Artificial Sequence
  Mab-1 Heavy Chain CDR Amino Acid Sequence
SYGMH

SEQ ID NO: 5 - protein, 17 aa, Artificial Sequence
  Mab-1 Heavy Chain CDR Amino Acid Sequence
YISSSGSTIYYADSVKG

SEQ ID NO: 6 - protein, 13 aa, Artificial Sequence
  Mab-1 Heavy Chain CDR Amino Acid Sequence
DLLDYDLLTGYGY

SEQ ID NO: 7 - protein, 112 aa, Artificial Sequence
  Mab-1 Variable Light Chain Amino Acid Sequence
DIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQGTHWPPTFGQGTKLEIK

SEQ ID NO: 8 - protein, 122 aa, Artificial Sequence
  Mab-1 Variable Heavy Chain Amino Acid Sequence
EVQLVQSGGGVVQPGRSLRLSCAASGFTFSSYGMHWVRQAPGKGLEWVSYISSSGSTIYYADSVKGRFTISRDNAKNSLYLQMNSLRAEDTAVYYCARDLLDYDLLTGYGYWGQGTLVTVSS

SEQ ID NO: 9 - protein, 241 aa, Artificial Sequence
  Mab-1 Light Chain Amino Acid Sequence
MDMRVPAQLLGLLLLWLRGARCDIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQGTHWPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 470 aa, Artificial Sequence
  Mab-1 Heavy Chain Amino Acid Sequence
MDMRVPAQLLGLLLLWLRGARCEVQLVQSGGGVVQPGRSLRLSCAASGFTFSSYGMHWVRQAPGKGLEWVSYISSSGSTIYYADSVKGRFTISRDNAKNSLYLQMNSLRAEDTAVYYCARDLLDYDLLTGYGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK